(19) United States
(12) Patent Application Publication
(10) Pub. No.: US 2016/0090602 A1
(43) Pub. Date: Mar. 31, 2016

(54) METHODS FOR PLANT TRANSFORMATION

(71) Applicant: BENSON HILL BIOSYSTEMS, INC., Research Triangle Park, NC (US)

(72) Inventor: Xingrong Wu, St. Charles, MO (US)

(73) Assignee: BENSON HILL BIOSYSTEMS, INC., Research Triangle Park, NC (US)

(21) Appl. No.: 14/863,532

(22) Filed: Sep. 24, 2015

Related U.S. Application Data
(60) Provisional application No. 62/055,189, filed on Sep. 25, 2014.

(51) Int. Cl. C12N 15/82

(52) U.S. Cl. CPC C12N 15/8205

(57) ABSTRACT

This invention relates to methods for the transformation of Brachypodium sp. and transformed plants produced according to the method. Specifically, this invention relates to direct transformation of callus derived from immature embryos using Agrobacterium-mediated transformation, and plants regenerated from the transformed callus tissue.

The methods comprise utilizing Brachypodium sp. immature embryos as the source of plant material for callus induction; induced calli can be infected by Agrobacterium hosting an appropriate binary vector. Transgenic plants are regenerated from transgenic calli grown under conditions favoring growth of transformed cells while substantially inhibiting growth of non-transformed cells. These methods provide for significantly increased plant transformation efficiency with minimal ratio of escapes.

### FIELD OF THE INVENTION

The invention is drawn to plant transformation, particularly to methods for the transformation of plant species from the genus Brachypodium.

### BACKGROUND OF THE INVENTION

Current protocols for Brachypodium distachyon transformation use callus derived from immature embryos as the target tissue for Agrobacterium-mediated transformation. Agrobacterium-mediated transformation is performed by co-cultivation of Agrobacterium cells harboring the transformation vector with the plant tissue to be transformed. After the Agrobacterium cells are substantially removed from the plant tissue, the plant tissue is then transferred to selection medium. This selection medium contains appropriate chemicals (e.g., antibiotics and/or herbicides) to select for transformed cells. Following selection, plant tissue is transferred to regeneration medium, where shoots are produced. These shoots are then transferred to rooting medium. Following root development, plantlets are then transferred to soil for cultivation.

Optimizing transformation protocols for a plant species requires optimizing the tissue culture response of the species to optimize the condition of the plant tissue to be transformed. Typically, a suitable tissue culture response has been obtained by optimizing medium components and/or explant material and source, and/or growing conditions. This has led to success to some extent, but it still takes a significant amount of effort to efficiently obtain a sufficient number of independent transgenic events quickly. It would save considerable time and money if genes could be more efficiently introduced. Accordingly, methods are needed in the art to increase transformation efficiencies of Brachypodium species including B. distachyon.

### SUMMARY OF THE INVENTION

The present invention provides an efficient, cost effective method for stably transforming Brachypodium distachyon, which is a widely recognized model C3 grass. This model plant species can serve as a gene discovery/validation platform for wheat, maize, and other agronomically and economically important crops.

Efficient protocols have also been established to produce and characterize T-DNA insertional mutants for the genotype Bd21 (e.g., Christiansen et al (2005) Plant Cell Rep 23:751-758; Vain et al (2008) Plant Biotechnol J 6:236-245; Vogel and Hill (2008) Plant Cell Rep 27:471-478; Pacurar et al (2008) Transgenic Res 17:955-963; Alves et al (2009) Nat Protocols 4:638-649; Thole and Vain (2012) in Transgenic Plants: Methods and Protocols, Methods in Molecular Biology 847:137-149), which is the sequenced genotype and focus of the present biological investigations. Improving the transformation efficiency of Brachypodium is important because large numbers of transgenic plants are needed to enable studies on the effect of a large number of candidate genes or gene combinations within a given period of time. The method of the present invention is less labor-intensive than currently available protocols, and provides optimized transformation protocols and tissue culture media compositions, thereby increasing the transformation efficiency and providing higher throughput.

Major changes for B. distachyon transformation as compared with previously published protocols include:
Growing Agrobacterium at lower temperature;
A simplified re-suspension procedure;
A modified Infection medium that helps to improve infection (increase T-DNA delivery rate and provide higher levels of transient GFP expression); and,
Using a reduced Agrobacterium suspension density that helps to avoid Agrobacterium overgrowth during selection.

These modifications reduced the amount of starting callus needed for a single transformation vector, hence improving the throughput. These modified protocols will require fewer working hours than currently established protocols to obtain the desired number of independent events.

### DETAILED DESCRIPTION OF THE INVENTION

Improved methods and systems for B. distachyon transformation and regeneration are provided herein. The examples below detail the application of these methods and systems. These improved methods result in significantly increased plant transformation efficiency as compared to previously established transformation protocols.

An increased “transformation efficiency,” as used herein, refers to any improvement, such as an increase in transformation frequency and quality of events that impact the overall efficiency of the transformation process by reducing the amount of resources required. Transformation efficiency can be calculated by dividing the number of transgenic events recovered from a given transformation experiment by the number of immature embryos used to produce the embryogenic callus that was used for said transformation experiment.

Although B. distachyon may be used as an effective model plant system for the study of agronomic traits, genetic transformation of B. distachyon has been difficult to perform with a high efficiency. There are a number of reports of B. distachyon in the scientific literature, with transformation efficiencies ranging from less than one transgenic event per immature embryo up to eleven transgenic events per immature embryo, as shown in Table One.

TABLE One
Reported B. distachyon transformation efficiencies

| | Transformation Efficiency |
|---|---|
| | (transgenic events |
| Source | per immature embryo) |
| Alves et al (2009) | 8 |
| Nat Protocols | |
| 4: 638-649. | |
| Pacurar et al (2008) | <0.55 |
| Transgenic Res | |
| 17: 955-963. | |
| Vain et al (2008) | 2.7 |
| Plant Biotechnol J | |
| 6: 236-245. | |
| Vogel and Hill (2008) | 11 |
| Plant Cell Rep | |
| 27: 471-478. | |
| Christiansen et al (2005) | Not reported |
| Plant Cell Rep | |
| 23: 751-758. | |
| Thole and Vain (2012) | Not reported |
| in Transgenic Plants: | |
| Methods and Protocols, | |
| Methods in Molecular | |
| Biology 847: 137-149. | |
| Present Invention | 15.75 |

The transformation protocols and methods of the present invention provide a transformation efficiency of at least 15 events per immature embryo, more specifically about 15.75 transgenic events per immature embryo. This is an increased transformation efficiency relative to the previously published methods of Brachypodium transformation.

The following examples are offered by way of illustration and not by way of limitation.

### Experimental

### Example 1

Plant materials for B. distachyon Callus Transformation

Plant materials: Tillers from 7-9 week old Brachypodium plants grown under 20-hour photoperiod
Agrobacterium strain: AGL-1 or LBA4404 harboring binary vector pMDC99/super binary vector pSB 1 with ZmUbi/D35S driving Hpt as plant selectable marker, and GFP as reporter gene

### Example 2

Media recipes for B. distachyon transformation

### YEP Medium:

5 g/L yeast extract, 10 g/L peptone, 5 g /L NaCl2, 15 g/L Bacto-agar. pH to 6.8 with NaOH. Appropriate antibiotics (Kanamycin stock at 50 mg/L) should be added to the medium when cooled to 50° C. after autoclaving.

### Brachy Callus Induction Medium (CIM):

4.33 g/L MS salt and MS vitamins, 30 g/L sucrose, 2.5 ml/L 2, 4-D (1 mg/ml), 8.0 g/L Agar.
Adjust with KOH to pH5.8, autoclave.

### Infection Medium:

2.16 g/L MS salt, 1 ml/L MS vitamins (1000×), 68.5 g/L sucrose, 36 g/L glucose, 0.115 g/L L-proline, 1.5 ml/L 2,4-D (1 mg/ml). Adjust with KOH to pH5.2, autoclave. Add 1 ml/L Acetosyringone (100 mM) before use.

### Selection Medium:

4.33 g/L MS salt and MS vitamins, 30 g/L sucrose, 2.5 ml/L 2, 4-D (1 mg/ml), 8.0 g/L Agar. Adjust with KOH to pH5.8, autoclave. Filter-sterilized 40 mg/L hygromycin, Timentin 100 mg/L, cefotaxime 150 mg/L cocktail is added prior to use.

### Regeneration Medium I

4.33 g/L MS salt and vitamins, 30 g/L sucrose, adjust with KOH to pH5.8, autoclave. Filter-sterilized 0.2 mg/L Kinetin, 20 mg/L hygromycin, Timentin 100 mg/L, cefotaxime 150 mg/L cocktail is added prior to use.

### Regeneration Medium II:

2.16 g/L MS Salts and vitamins, 30 g /L sucrose, 2.6 g/L Phytogel (pH 5.8).

### MSB3+Cu0.6:

Dissolve 100 ml of Macro MS salts stock solution (10×), 10 ml of Micro MS salts stock solution (100×), 10 ml of Fe-EDTA stock solution (100×), 30 g of sucrose, 2.5 ml of 2,4-D (1 mg/ml) in 990 ml of deionized water. Add 2 g of Phytagel and adjust pH to 5.8 using KOH. Add 600 μL CuSO₄ (1 mg/mL). After autoclaving, let the medium cool down and add 10 ml of filter-sterilized M5 vitamins stock solution (100×).

### Example 3

Methods for Brachypodium transformation

1. Collect tillers from 7- to 9-week-old Bd21 plants grown in a controlled environment room at 22° C. with a 20-hour photoperiod when the immature seeds are swollen but still green.

2. Select immature seeds with a soft endosperm, remove lemma, sterilize and rinse. To perform this step, collect the seeds in water and then drain well before sterilization. Sterilize approximately 20 seeds for 30 seconds with 20 mL of 70% ethanol in a sterile Petri dish with a lid. Drain ethanol and rinse with sterile deionized water. Add 20 mL of 1.3% sodium hypochlorite solution. Gently shake the seeds on a tabletop orbital shaker for 4 minutes. Rinse three times with sterile deionized water.

3. Isolate immature embryos up to and including 0.3 mm in length from seeds using fine forceps and a stereomicroscope under sterile conditions (i.e., in a laminar flow hood).

4. Culture immature embryos (10-20 per plate), with the scutellum facing up, onto MSB3 +Cu0.6 solid medium for 3 weeks at 25° C. in the dark.

5. Excise the shoots under sterile conditions, as they elongate during the first 2-3 days of culture.

6. At week 3 (i.e., 3 weeks after step 4), fragment compact embryogenic callus (CEC) with a creamy color and pearly surface in 1-3 pieces. Transfer the pieces of CEC onto fresh MSB3+Cu0.6 solid medium (16-20 calli per plate) for another 2 weeks at 25° C. in the dark. Discard all non-CEC tissue.

7. At week 5 (i.e., 2 weeks after step 6), split CEC with a creamy color and pearly surface in 4-6 pieces. Transfer pieces of CEC onto fresh MSB3+Cu0.6 solid medium (16-20 calli per plate) for another week at 25° C. in the dark. Discard all non-CEC tissue.

8. At week 6 (i.e., 1 week after step 7 and on the day of transformation), split CEC one last time in 4-6 pieces and place 50-100 CEC pieces on fresh MSB3+Cu0.6 solid medium before inoculation with Agrobacterium.

9. Transfer dark-growing calli to CIM medium at 28° C. for three to eleven days.

10. Agrobacterium cultures are grown for three days at 19 to 22° C. on YEP medium amended with 50 mg/L kanamycin.

11. A small amount of bacterial culture is scraped from the plate and suspended in approximately 15 ml of liquid infection medium supplemented with 100 μM acetosyringone in a 50 ml conical tube. Adjust the optical density to less than OD₆₀₀=0.25 before use. Typically an OD₆₀₀ of 0.15 is used.

12. For each construct, transfer a small amount of actively growing calli to a tube. Using sterile forceps, subculture compact calli from their original plates and transfer them to their corresponding square petri dish. Try to make them a reasonable size as if they are too small, they will not survive the transformation.

13. Add 6 ml Agrobacterium suspension and allow calli to incubate in culture at room temperature (i.e., 25-35° C.) for 5-7 minutes in the dark.

14. Extract the Agrobacterium culture from the dish. Extract the cultures in the same order that poured the culture onto the calli. This will allow the incubation time to be even. Use a serological pipette to remove the majority of the culture. You can then use a regular pipette to survey the plate and to slowly extract any remaining culture

15. Making sure that all of the culture has been extracted, transfer calli to their respective filter paper plates. The filter paper should be on the top lid of the petri dish.

16. Allow infected calli to air-dry in the flow hood for 3-6 minutes until no major trace of liquid is visible.

17. Close all plates and carefully wrap plates in Parafilm. Calli will either break or adhere to top of petri dish if they are not handled with care. Co-cultivation plates are incubated in the dark at 25° C. for three days.

18. Transfer infected calli from the filter paper to the selection medium.

19. Selection plates are wrapped in Parafilm and placed in the dark at 28° C.

20. Every two weeks, the tissue is sub-cultured onto fresh selection medium. This should be done with the aid of a microscope to look for any evidence of Agrobacterium overgrowth. If overgrowth is noted, the affected calli should be avoided (do not transfer contaminated calli). The remaining tissue (unaffected by overgrowth) can then be carefully transferred using a newly sterilized forceps for each callus piece.

21. Putative clones begin to appear after six to eight weeks on selection. A clone is recognized as white, actively growing callus and is distinguishable from the brown, unhealthy non-transformed tissue. Individual transgenic events are identified and the white, actively growing tissue is transferred to a new plate in order to produce enough tissue for the next step.

22. Transfer active growing calli to regeneration/selection plates for shoot induction at 28° C. in light growth chamber until shoots become excisable.

23. Remove shoots from calli with forceps and transfer them to Regeneration Medium II for rooting at 28° C. and 16/8 photoperiods with proper light intensity.

### Example 4

Brachypodium Transformation Results

Transformations were performed according to the protocols described above. Four immature embryos were used for the production of CEC. Following transformation and regeneration of transformed events, 63 confirmed transgenic events were confirmed by the presence of a GFP-derived fluorescence signal that was not present in wild-type Brachypodium plants. Thus, the transformation protocol described herein resulted in a transformation efficiency of greater than 15 events per immature embryo. Specifically, about 15.75 events per immature embryo were achieved using this protocol.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

We claim:

1. A method of increasing the transformation frequency for transforming callus derived from immature embryos of Brachypodium sp. comprising:
(a) Producing embryogenic callus from immature embryos of Brachypodium sp.,
(b) Growing Agrobacterium cells for three days at a temperature of 19-22° C. harboring a functional plant transformation vector,
(c) Re-suspending the Agrobacterium cells in infection medium to an optical density of less than 0.25 at 600 nm,
(d) co-cultivating the resuspended Agrobacterium cells with callus tissue in infection medium containing greater than 30 g/L sucrose,
(e) culturing the infected cell on selection medium at a temperature of 25-35° C. in the dark to produce transformed tissue expressing the nucleic acid,
(f) regenerating the transformed tissue on at least one regeneration medium to produce a transformed plant,
wherein the resulting transformation efficiency is at least 15 transgenic events per immature embryo.

2. The method of claim 1, where the plant cells being transformed are derived from Brachypodium distachyon.

3. The method of claim 1, where the Agrobacterium cells are resuspended in infection medium at an optical density of 0.10-0.20.

4. The method of claim 1, where the Agrobacterium cells are resuspended in infection medium at an optical density of 0.13-0.17.

5. The method of claim 1, where the Agrobacterium cells are resuspended in infection medium at an optical density of 0.14-0.16.

6. The method of claim 1, where the Agrobacterium cells are resuspended in infection medium at an optical density of 0.15.
